(19) United States
(12) Patent Application Publication
(10) Pub. No.: US 2016/0082247 A1
(43) Pub. Date: Mar. 24, 2016

(54) SYSTEMS AND METHODS FOR MAKING AND USING ANCHORING ARRANGEMENTS FOR LEADS OF ELECTRICAL STIMULATION SYSTEMS

(71) Applicant: Boston Scientific Neuromodulation Corporation, Valencia, CA (US)

(72) Inventors: James Robert Black, Medina, OH (US); David Ernest Wechter, Santa Clarita, CA (US)

(21) Appl. No.: 14/861,713

(22) Filed: Sep. 22, 2015

Related U.S. Application Data
(60) Provisional application No. 62/053,492, filed on Sep. 22, 2014.

(51) Int. Cl. A61N 1/05

(52) U.S. Cl. CPC A61N 1/05

(57) ABSTRACT

A lead assembly includes an electrical stimulation lead with a lead body having a distal portion, a proximal portion, a longitudinal length, and an outer surface. At least one electrode is disposed along the distal portion of the lead body. At least one terminal is disposed along the proximal portion of the lead body. At least one lead conductor electrically couples the at least one electrode to the at least one terminal. An anchoring arrangement is configured and arranged to reduce undesired movement of the lead relative to a patient when the lead is inserted into the patient. The anchoring arrangement includes at least one helical member attached to, and projecting outwardly from, the outer surface of the lead. The at least one helical member extends along at least 30% of the longitudinal length of the lead and makes at least one full coil around the lead.

### CROSS-REFERENCE TO RELATED APPLICATIONS

This application claims the benefit under 35 U.S.C. §119(e) of U.S. Provisional Patent Application Ser. No. 62/053,492 filed Sep. 22, 2014, which is incorporated herein by reference.

### FIELD

The present invention is directed to the area of implantable electrical stimulation systems and methods of making and using the systems. The present invention is also directed to anchoring arrangements for passively anchoring implantable electrical stimulation leads, as well as methods of making and using the leads, anchoring arrangements, and electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

In one embodiment, a lead assembly includes an electrical stimulation lead with a lead body having a distal portion, a proximal portion, a longitudinal length, and an outer surface. At least one electrode is disposed along the distal portion of the lead body. At least one terminal is disposed along the proximal portion of the lead body. At least one lead conductor electrically couples the at least one electrode to the at least one terminal. An anchoring arrangement is configured and arranged to reduce undesired movement of the lead relative to a patient when the lead is inserted into the patient. The anchoring arrangement includes at least one helical member attached to, and projecting outwardly from, the outer surface of the lead. The at least one helical member extends along at least 30% of the longitudinal length of the lead and makes at least one full coil around the lead.

In at least some embodiments, the at least one helical member extends along the entire longitudinal length of the lead between the at least one electrode and the at least one terminal. In at least some embodiments, the at least one helical member extends exclusively along the distal portion of the lead.

In at least some embodiments, the at least one helical member is formed entirely from electrically nonconductive polymer. In at least some embodiments, the at least one helical member includes a first anchor conductor encased in electrically nonconductive material. In at least some embodiments, the at least one electrode includes a first electrode and a second electrode, wherein the at least one terminal comprises a first terminal and a second terminal, wherein the first electrode is coupled to the first terminal via the at least one lead conductor, and wherein the second electrode is coupled to the second terminal via the first anchor conductor.

In at least some embodiments, the at least one helical member further includes a second anchor conductor encased in the electrically nonconductive material. In at least some embodiments, the at least one helical member includes a single helical member, and wherein the first anchor conductor and the second anchor conductor are both disposed along the single helical member. In at least some embodiments, the at least one helical member includes a first helical member and a second helical member, and wherein the first anchor conductor is disposed along the first helical member and the second anchor conductor is disposed along the second helical member. In at least some embodiments, the at least one electrode further includes a third electrode and the at least one terminal further includes a third terminal, and wherein the second anchor conductor couples the third electrode to the third terminal.

In at least some embodiments, the at least one helical member has a constant pitch. In at least some embodiments, the at least one helical member has a variable pitch.

In at least some embodiments, the anchoring arrangement further includes a meshed material disposed along a portion of the longitudinal length of the lead body. In at least some embodiments, the anchoring arrangement further includes at least one of an outwardly-projecting knob or a longitudinal strip attached to, and extending along, the outer surface of the lead.

In at least some embodiments, an electrical stimulating system includes the above-described lead assembly, a control module, and a connector. The control module is coupleable to the electrical stimulation lead of the lead assembly. The control module includes a housing, and an electronic subassembly disposed in the housing. The connector receives the electrical stimulation lead. The connector includes a connector housing defining a port configured and arranged for receiving the proximal portion of the lead body of the electrical stimulation lead. The connector also includes at least one connector contact disposed in the connector housing. The at least one connector contact is configured and arranged to couple to the at least one terminal of the electrical stimulation lead when the proximal portion of the electrical stimulation lead is received by the port.

In a further embodiment, a method of forming the electrical stimulation lead includes: disposing a plurality of electrodes along a distal portion of a lead body, the plurality of electrodes comprising a first electrode; disposing a plurality of terminals along a proximal portion of the lead body, the plurality of terminals comprising a first terminal; electrically coupling the first electrode to the first terminal using a lead conductor extending along a longitudinal length of the lead within an outer surface of the lead; and attaching a helical member to the outer surface of the lead, the helical member extending along at least 30% of the longitudinal length of the lead and making at least one full coil around the lead.

In at least some embodiments, attaching helical member to the outer surface of the lead includes at least one of reflowing or chemically bonding the helical member to the lead. In at least some embodiments, the above method further includes electrically coupling a second electrode of the plurality of electrodes to a second terminal of the plurality of terminals using an anchor conductor disposed in the helical member.

In another embodiment, a lead assembly includes an electrical stimulation lead with a lead body having a distal portion, a proximal portion, a longitudinal length, and an outer surface. At least one electrode is disposed along the distal portion of the lead. At least one terminal is disposed along the proximal portion of the lead. At least one lead conductor electrically couples the at least one electrode to the at least one terminal. An anchoring arrangement is configured and arranged to reduce undesired movement of the lead relative to a patient when the lead is inserted into the patient. The anchoring arrangement includes a plurality of anchoring elements attached to, and projecting outwardly from, the outer surface of the lead. The plurality of anchoring elements includes a proximal-most element, a distal-most element, and a plurality of intermediate elements disposed between the proximal-most element and the distal-most element. At least 30% of the longitudinal length of the lead body is disposed between the proximal-most element and the distal-most element. The plurality of anchoring elements include at least one of a ring, a strip, or a knob.

In yet another embodiment, a lead assembly includes an electrical stimulation lead with a lead body having a distal portion, a proximal portion, a longitudinal length, and an outer surface. At least one electrode is disposed along the distal portion of the lead. At least one terminal is disposed along the proximal portion of the lead. At least one lead conductor electrically couples the at least one electrode to the at least one terminal. An anchoring arrangement is configured and arranged to reduce undesired movement of the lead relative to a patient when the lead is inserted into the patient. The anchoring arrangement includes a meshed material disposed over, and attached to, the outer surface of the lead. The meshed material extends along at least 30% of the longitudinal length of the lead.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system that includes a lead electrically coupled to an implantable control module, according to the invention;

FIG. 2A is a schematic view of one embodiment of the control module of FIG. 1 configured and arranged to electrically couple to an elongated device, according to the invention;

FIG. 2B is a schematic view of one embodiment of a lead extension configured and arranged to electrically couple the elongated device of FIG. 2A to the control module of FIG. 1, according to the invention;

FIG. 3 is a schematic view of one embodiment of a trial stimulation system that includes a lead that is at least partially inserted into a patient and is coupleable to an external trial stimulator, according to the invention;

FIG. 4 is a schematic side view of one embodiment of the lead of FIG. 3, the lead including a single electrode coupled to a single terminal, according to the invention;

FIG. 5A is a schematic side view of one embodiment of an anchoring arrangement disposed along the lead of FIG. 3, the anchoring arrangement including a helical member extending along an outer surface of the lead, according to the invention;

FIG. 5B is a schematic side view of another embodiment of the anchoring arrangement of FIG. 5A disposed along the lead of FIG. 3, the lead including an electrode and a terminal coupled to one another by an anchor conductor of the anchoring arrangement, according to the invention;

FIG. 5C is a schematic side view of yet another embodiment of the anchoring arrangement of FIG. 5A disposed along the lead of FIG. 3, the lead including first and second electrodes and first and second terminals, the first electrode coupled to the first terminal by a lead conductor disposed within the lead, the second electrode coupled to the second terminal by an anchor conductor of the anchoring arrangement, according to the invention;

FIG. 6A is a schematic side view of another embodiment of the anchoring arrangement of FIG. 5A disposed along a lead with three electrodes coupled to three terminals, the anchoring arrangement including a helical member extending along an outer surface of the lead, according to the invention;

FIG. 6B is a schematic side view of yet another embodiment of the anchoring arrangement of FIG. 5A disposed along the lead of FIG. 6A, the anchoring arrangement including an anchor conductor electrically coupling one of the three electrodes to one of the three terminals, according to the invention;

FIG. 6C is a schematic side view of another embodiment of the anchoring arrangement of FIG. 5A disposed along the lead of FIG. 6A, the anchoring arrangement including two anchor conductors disposed along a single helical member, each of the two anchor conductors electrically coupling a different electrode of the three electrodes to a different terminal of the three terminals, according to the invention;

FIG. 6D is a schematic side view of yet another embodiment of the anchoring arrangement of FIG. 5A disposed along the lead of FIG. 6A, the anchoring arrangement including two helical members extending along an outer surface of the lead, each helical member including a different anchor conductor electrically coupling a different one of the three electrodes to a different one of the three terminals, according to the invention;

FIG. 7A is a schematic side view of another embodiment of the anchoring arrangement of FIG. 5A disposed along a portion of a lead, the anchoring arrangement including multiple longitudinally-spaced-apart rings disposed around a circumference of the lead, according to the invention;

FIG. 7B is a schematic side view of yet another embodiment of the anchoring arrangement of FIG. 5A disposed along a portion of the lead of FIG. 7A, the anchoring arrangement including multiple circumferentially-spaced-apart longitudinal strips disposed along a longitudinal length of the lead, according to the invention;

FIG. 7C is a schematic side view of still yet another embodiment of the anchoring arrangement of FIG. 5A disposed along a portion of the lead of FIG. 7A, the anchoring arrangement including multiple outwardly-projecting knobs disposed along an outer surface of the lead, according to the invention;

FIG. 7D is a schematic side view of another embodiment of the anchoring arrangement of FIG. 5A disposed along a portion of the lead of FIG. 7A, the anchoring arrangement including a meshed material disposed over a portion of an outer surface of the lead, according to the invention;

FIG. 8 is a schematic view of yet another embodiment of the anchoring arrangement of FIG. 5A disposed along a lead, the anchoring arrangement including a helical member extending along an outer surface of the lead, the helical member wrapping around a circumference of the lead in multiple different pitches, according to the invention;

FIG. 9 is a schematic view of another embodiment of the anchoring arrangement of FIG. 5A disposed along the lead of FIG. 8, the anchoring arrangement including a helical member extending along an outer surface of the lead, the helical member extending exclusively along a distal portion of the lead, according to the invention;

FIG. 10 is a schematic view of yet another embodiment of the anchoring arrangement of FIG. 5A disposed along a lead, the anchoring arrangement including the helical member of FIG. 9, the longitudinally-spaced-apart rings of FIG. 7A, and the meshed material of FIG. 7D, according to the invention; and

FIG. 11 is a schematic overview of one embodiment of components of a stimulation system, including an electronic subassembly disposed within a control module, according to the invention.

### DETAILED DESCRIPTION

The present invention is directed to the area of implantable electrical stimulation systems and methods of making and using the systems. The present invention is also directed to anchoring arrangements for passively anchoring implantable electrical stimulation leads, as well as methods of making and using the leads, anchoring arrangements, and electrical stimulation systems.

Suitable implantable electrical stimulation systems include, but are not limited to, a least one lead with one or more electrodes disposed along a distal end of the lead and one or more terminals disposed along the one or more proximal ends of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,949,395; 7,244,150; 7,672,734; 7,761,165; 7,974,706; 8,175,710; 8,224,450; and 8,364,278; and U.S. Patent Application Publication No. 2007/0150036, all of which are incorporated by reference.

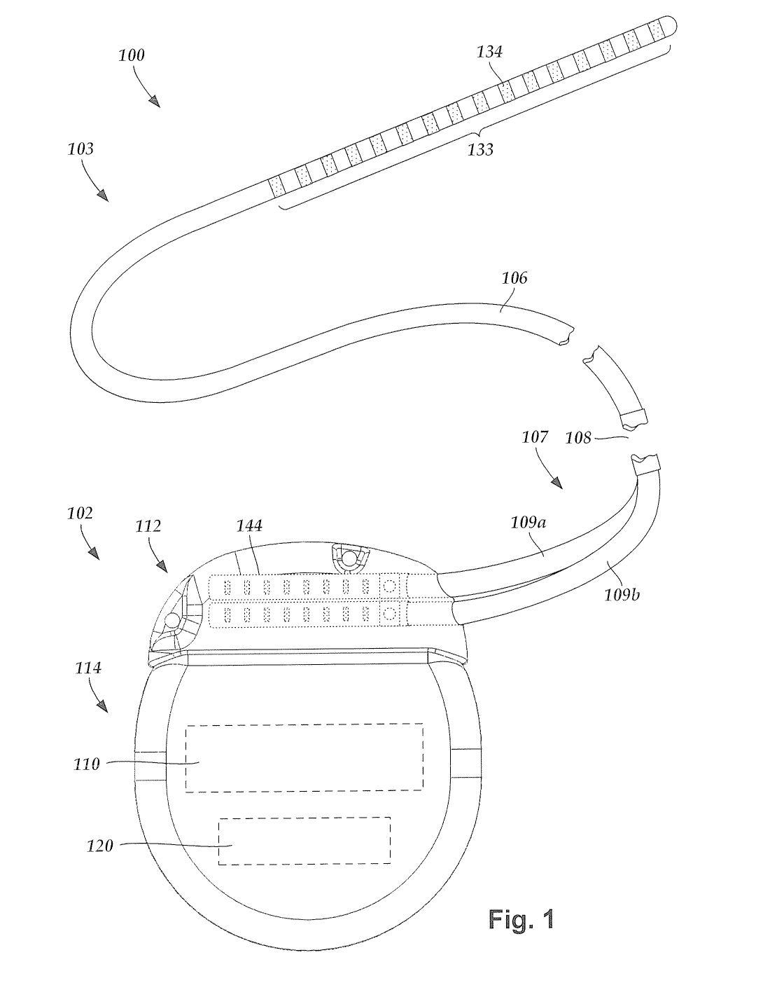

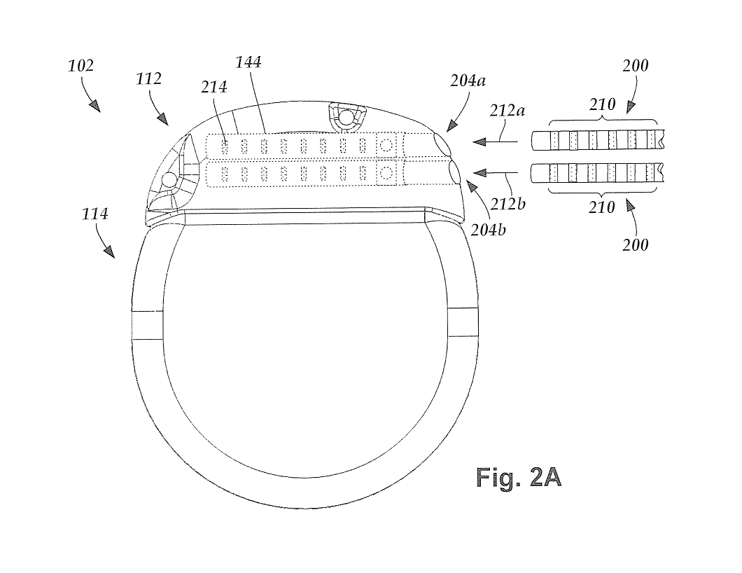

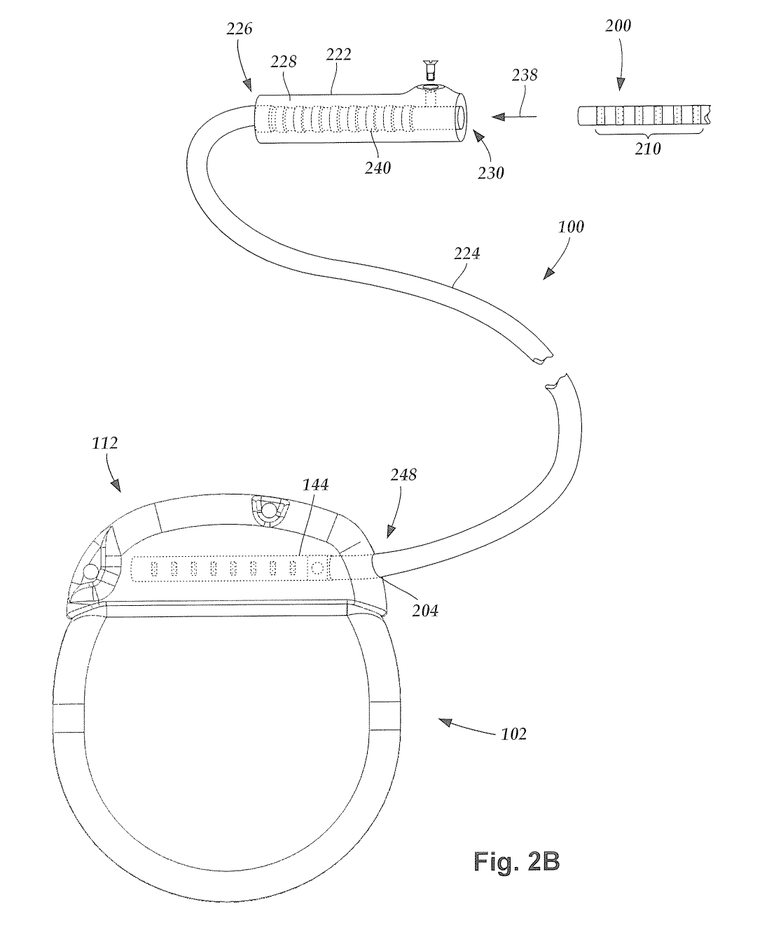

FIG. 1 illustrates schematically one embodiment of an electrical stimulation system 100. The electrical stimulation system includes a control module (e.g., a stimulator or pulse generator) 102 and a lead 103 coupleable to the control module 102. The lead 103 includes one or more lead bodies 106, an array of electrodes 133, such as electrode 134, and an array of terminals (e.g., 210 in FIG. 2A-2B) disposed along the one or more lead bodies 106. In at least some embodiments, the lead is isodiametric along a longitudinal length of the lead body 106.

The lead 103 can be coupled to the control module 102 in any suitable manner. In at least some embodiments, the lead 103 couples directly to the control module 102. In at least some other embodiments, the lead 103 couples to the control module 102 via one or more intermediate devices (200 in FIGS. 2A-2B). For example, in at least some embodiments one or more lead extensions 224 (see e.g., FIG. 2B) can be disposed between the lead 103 and the control module 102 to extend the distance between the lead 103 and the control module 102. Other intermediate devices may be used in addition to, or in lieu of, one or more lead extensions including, for example, a splitter, an adaptor, or the like or combinations thereof. It will be understood that, in the case where the electrical stimulation system 100 includes multiple elongated devices disposed between the lead 103 and the control module 102, the intermediate devices may be configured into any suitable arrangement.

In FIG. 1, the electrical stimulation system 100 is shown having a splitter 107 configured and arranged for facilitating coupling of the lead 103 to the control module 102. The splitter 107 includes a splitter connector 108 configured to couple to a proximal end of the lead 103, and one or more splitter tails 109a and 109b configured and arranged to couple to the control module 102 (or another splitter, a lead extension, an adaptor, or the like).

The control module 102 typically includes a connector housing 112 (e.g., a header) and a sealed electronics housing 114. An electronic subassembly 110 and an optional power source 120 are disposed in the electronics housing 114. A control module connector 144 is disposed in the connector housing 112. The control module connector 144 is configured and arranged to make an electrical connection between the lead 103 and the electronic subassembly 110 of the control module 102.

The electrical stimulation system or components of the electrical stimulation system, including one or more of the lead bodies 106 and the control module 102, are typically implanted into the body of a patient. The electrical stimulation system can be used for a variety of applications including, but not limited to, brain stimulation, neural stimulation, spinal cord stimulation, muscle stimulation, and the like.

The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, or titanium. The number of electrodes 134 in each array 133 may vary. For example, there can be two, four, six, eight, ten, twelve, fourteen, sixteen, or more electrodes 134. As will be recognized, other numbers of electrodes 134 may also be used.

The electrodes of the one or more lead bodies 106 are typically disposed in, or separated by, a non-conductive, biocompatible material such as, for example, silicone, polyurethane, polyetheretherketone (“PEEK”), epoxy, and the like or combinations thereof The lead bodies 106 may be formed in the desired shape by any process including, for example, molding (including injection molding), casting, and the like. The non-conductive material typically extends from the distal end of the one or more lead bodies 106 to the proximal end of each of the one or more lead bodies 106.

Terminals (e.g., 210 in FIGS. 2A-2B) are typically disposed along the proximal end of the one or more lead bodies 106 of the electrical stimulation system 100 (as well as any splitters, lead extensions, adaptors, or the like) for electrical connection to corresponding connector contacts (e.g., 214 in FIGS. 2A-2B; and 240 in FIG. 2B). The connector contacts are disposed in connectors (e.g., 144 in FIGS. 1-2B; and 222 in FIG. 2B) which, in turn, are disposed on, for example, the control module 102 (or a lead extension, a splitter, an adaptor, or the like). Electrically conductive wires, cables, or the like (not shown) extend from the terminals to the electrodes 134. Typically, one or more electrodes 134 are electrically coupled to each terminal. In at least some embodiments, each terminal is only connected to one electrode 134.

The electrically conductive wires (“conductors”) may be embedded in the non-conductive material of the lead body 106 or can be disposed in one or more lumens (not shown) extending along the lead body 106. In some embodiments, there is an individual lumen for each conductor. In other embodiments, two or more conductors extend through a lumen. There may also be one or more lumens (not shown) that open at, or near, the proximal end of the lead body 106, for example, for inserting a stylet to facilitate placement of the lead body 106 within a body of a patient. Additionally, there may be one or more lumens (not shown) that open at, or near, the distal end of the lead body 106, for example, for infusion of drugs or medication into the site of implantation of the one or more lead bodies 106. In at least one embodiment, the one or more lumens are flushed continually, or on a regular basis, with saline, epidural fluid, or the like. In at least some embodiments, the one or more lumens are permanently or removably sealable at the distal end.

FIG. 2A is a schematic side view of one embodiment of a proximal end of one or more elongated devices 200 configured and arranged for coupling to one embodiment of the control module connector 144. The one or more elongated devices may include, for example, the lead body 106, one or more intermediate devices (e.g., the splitter 107 of FIG. 1, the lead extension 224 of FIG. 2B, an adaptor, or the like or combinations thereof), or a combination thereof.

The control module connector 144 defines at least one port into which a proximal end of the elongated device 200 can be inserted, as shown by directional arrows 212a and 212b. In FIG. 2A (and in other figures), the connector housing 112 is shown having two ports 204a and 204b. The connector housing 112 can define any suitable number of ports including, for example, one, two, three, four, five, six, seven, eight, or more ports.

The control module connector 144 also includes a plurality of connector contacts, such as connector contact 214 disposed within each port 204a and 204b. When the elongated device 200 is inserted into the ports 204a and 204b, the connector contacts 214 can be aligned with a plurality of terminals 210 disposed along the proximal end(s) of the elongated device(s) 200 to electrically couple the control module 102 to the electrodes (134 of FIG. 1) disposed at a distal end of the lead 103. Examples of connectors in control modules are found in, for example, U.S. Pat. No. 7,244,150 and 8,224,450, which are incorporated by reference.

FIG. 2B is a schematic side view of another embodiment of the electrical stimulation system 100. The electrical stimulation system 100 includes a lead extension 224 that is configured and arranged to couple one or more elongated devices 200 (e.g., the lead body 106, the splitter 107, an adaptor, another lead extension, or the like or combinations thereof) to the control module 102. In FIG. 2B, the lead extension 224 is shown coupled to a single port 204 defined in the control module connector 144. Additionally, the lead extension 224 is shown configured and arranged to couple to a single elongated device 200. In alternate embodiments, the lead extension 224 is configured and arranged to couple to multiple ports 204 defined in the control module connector 144, or to receive multiple elongated devices 200, or both.

A lead extension connector 222 is disposed on the lead extension 224. In FIG. 2B, the lead extension connector 222 is shown disposed at a distal end 226 of the lead extension 224. The lead extension connector 222 includes a connector housing 228. The connector housing 228 defines at least one port 230 into which terminals 210 of the elongated device 200 can be inserted, as shown by directional arrow 238. The connector housing 228 also includes a plurality of connector contacts, such as connector contact 240. When the elongated device 200 is inserted into the port 230, the connector contacts 240 disposed in the connector housing 228 can be aligned with the terminals 210 of the elongated device 200 to electrically couple the lead extension 224 to the electrodes (134 of FIG. 1) disposed along the lead (103 in FIG. 1).

In at least some embodiments, the proximal end of the lead extension 224 is similarly configured and arranged as a proximal end of the lead 103 (or other elongated device 200). The lead extension 224 may include a plurality of electrically conductive wires (not shown) that electrically couple the connector contacts 240 to a proximal end 248 of the lead extension 224 that is opposite to the distal end 226. In at least some embodiments, the conductive wires disposed in the lead extension 224 can be electrically coupled to a plurality of terminals (not shown) disposed along the proximal end 248 of the lead extension 224. In at least some embodiments, the proximal end 248 of the lead extension 224 is configured and arranged for insertion into a connector disposed in another lead extension (or another intermediate device). In other embodiments (and as shown in FIG. 2B), the proximal end 248 of the lead extension 224 is configured and arranged for insertion into the control module connector 144.

Turning to FIG. 3, providing therapy using electrical stimulation may be a long-term process. Consequently, many conventional stimulation systems provide stimulation (via one or more implanted leads) of the patient over an extended period of time, such as the operational lifetime of the system, the remaining lifetime of the patient, or at least 0.5, 1, 5, 10, 15, 20, or more years.

In some instances, the potential efficacy of electrical stimulation for a particular patient is tested prior to implantation. One way to test efficacy is to perform a trial stimulation (e.g., a percutaneous nerve evaluation, or the like), whereby an electrode-including distal portion of a lead (and, optionally, one or more lead extensions) is temporarily inserted into the patient. The proximal portion of the lead (or lead extension) can then be electrically coupled to a trial stimulator that is disposed external to the patient to perform trial stimulations using the one or more electrodes. Once efficacy is established, the trial stimulation system can be removed and replaced with an implantable lead and control module (see e.g., FIG. 1).

The trial stimulations may continue for a short period (e.g., 3-10 days) where the patient is sent home with the trial stimulation system to assess the effectiveness of the therapy to determine if a permanent implanted system will be effective in treating the medical condition. During the trial stimulations, the proximal portion of the lead (or the proximal portion of a lead extension coupled to the lead) can be coupled directly to the trial stimulation. Alternately, the lead can be coupled to the trial stimulator by coupling the proximal portion of the lead (or the proximal portion of a lead extension coupled to the lead) to an operating room cable (“cable”) that, in turn, is electrically coupled to the trial stimulator.

FIG. 3 is a schematic view of one embodiment of a trial stimulation system 300 that includes a lead 303, an external trial stimulator 304, and one or more cables 306 that couple the lead 303 to the external trial stimulator 304. The lead 303 includes one or more electrodes 334 and one or more terminals 344. During operation, the electrode(s) 334 are disposed internal to the patient, while the terminal(s) 344 remain external to the patient, as shown in FIG. 3 by a line 320 schematically representing patient skin. In alternate embodiments, the lead may be coupled to a lead extension, where the entire lead and a distal portion of the lead extension are disposed in the patient while lead extension terminals remain external to the patient.

The terminal(s) 312 are configured and arranged to couple the electrode(s) 334 to the external trial stimulator 304. In at least some embodiments, a lead connector 322 of the cable 306 is configured and arranged to couple to the terminal(s) 344 of the lead 303 (or lead extension) and a trial stimulator connector 324 of the cable 306 is configured and arranged to couple to the external trial stimulator 304.

Turning to FIG. 4, any suitable type of lead configured for short-term (e.g., 3-10 days) insertion into a patient may be used for performing a trial stimulation. The leads may be inserted into the patient using any suitable technique including, for example, using a stylet, an introducer needle, or both. The leads may include any suitable number of electrodes and terminals. In some instances, for example, when the trial stimulation is configured for sacral nerve stimulation, the lead may include a single electrode coupled to a single terminal.

FIG. 4 illustrates, in schematic side view, one embodiment of the lead 303 suitable for use in a trial stimulation system (see e.g., 300 in FIG. 3). The lead 303 has a body 406 with an outer surface 408, a distal portion 410, and an opposing proximal portion 412. The lead 303 includes a single electrode 334 disposed along the distal portion 410 of the lead 303 and coupled to a single terminal 344 disposed along the proximal portion 412 of the lead 303. The electrode 334 is coupled to the terminal 344 via a lead conductor 454 disposed within the body 406 of the lead 303. In at least some embodiments, the entire portion of the lead conductor 454 extending between the electrode 334 and the terminal 344 is disposed beneath the outer surface 408 of the lead 303.

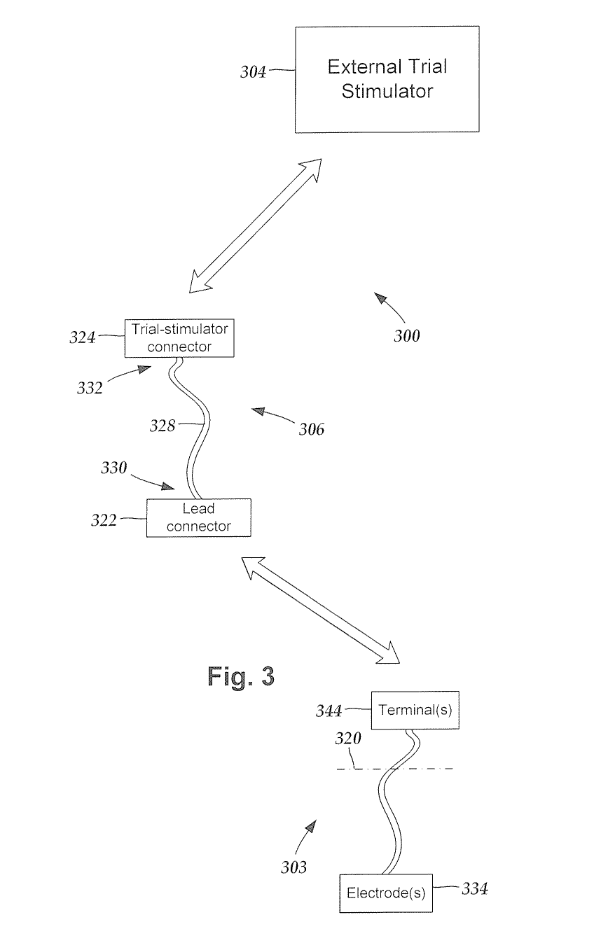

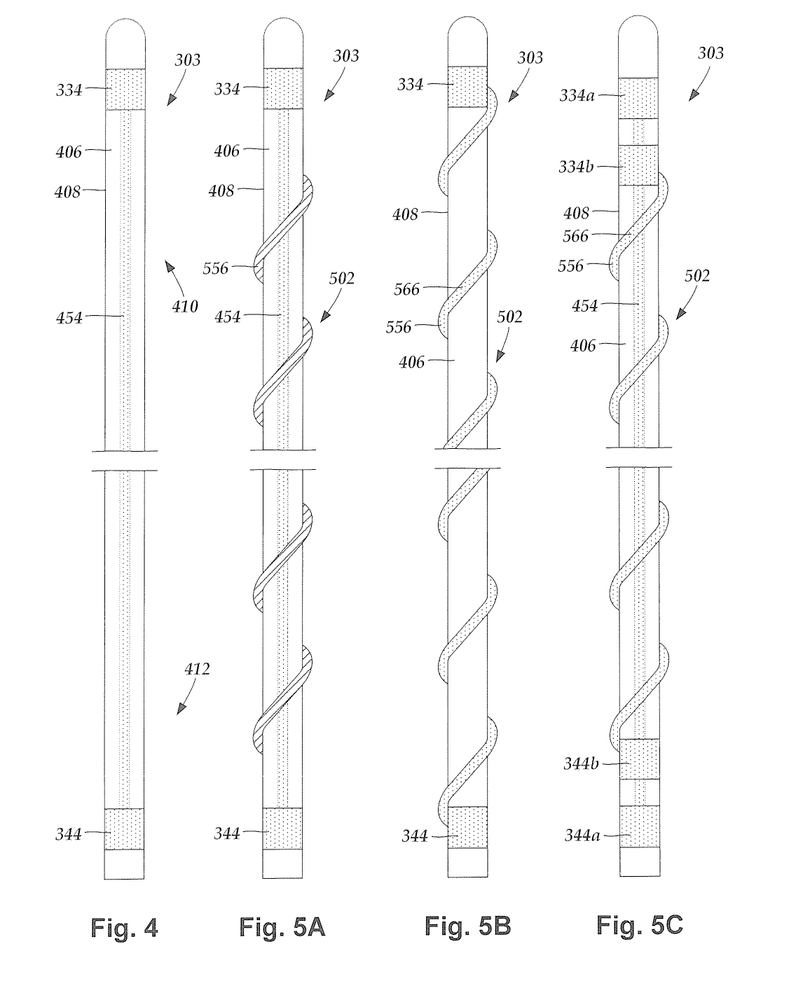

Turning to FIG. 5A, in some instances lead migration (i.e., undesired movement of the lead relative to the patient) may reduce, or even completely remove, the efficacy of electrical stimulation by increasing the distance between one or more electrodes and the target stimulation tissue. The increased distance between the one or more electrodes and the target stimulation tissue may prevent the stimulation from eliciting a desired response in the target stimulation tissue without increasing the amplitude of the stimulation to a level that may cause damage to other nearby tissue. Lead migration may be a particular problem when leads are inserted, or implanted, in regions of the body that are subjected to significant loading, such as leads adapted for sacral stimulation.

In the case of trial stimulations, lead migration may cause the trial stimulation to be unsuccessful and potentially prevent the implant candidate from receiving an implanted device. In the case of an implanted lead, lead migration may result in an undesired pr mature explantation of the lead.

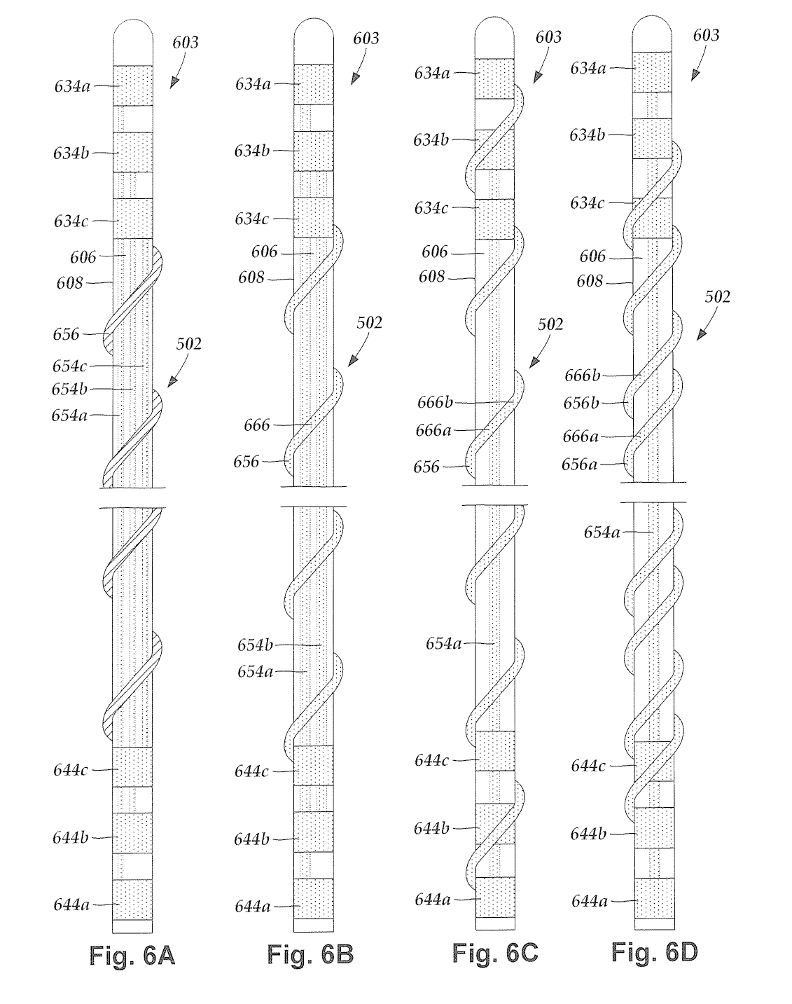

As herein described, an anchoring arrangement is used to passively (e.g., non-invasively) reduce, or even prevent, migration of an inserted (see e.g., 303 in FIGS. 4-5C and 8-10), or implanted (see e.g., 103 in FIG. 1; 603 in FIGS. 6A-6D), lead. The anchoring arrangement is attached to an outer surface of the lead and includes one or more features that project outwardly from the lead, thereby increasing the surface area of the lead in contact with patient tissue, when inserted, or implanted, into the patient, as compared with comparably-sized leads having round transverse cross-sections. In at least some embodiments, the anchoring arrangement is melded to the lead. The increased surface area may add resistance when axial tension is applied with the expanded transverse cross-sectional profile. The increased resistance may reduce, or even prevent, undesired lead migration while the lead is inserted, or implanted, into the patient.

In some embodiments, the one or more features of the anchoring arrangement include one or more helical members that form at least one coil around the lead. The helical member can coil around the lead any suitable number of times including, for example, one, two, three, four, five, six, seven, eight, nine, ten, eleven, twelve, fifteen. twenty, thirty, forty, fifty, or more times.

FIG. 5A illustrates, in schematic side view, one embodiment of an anchoring arrangement 502 disposed along the trial stimulation lead 303. The anchoring arrangement 502 includes a helical member 556 extending along the outer surface 408 of the lead 303. In at least some embodiments, the helical member 556 is formed from an electrically nonconductive polymer. In at least some embodiments, the helical member 556 is formed from an electrically nonconductive polymer (e.g., polyurethane, polytetrafluoroethylene, polyethylene terephthalate, polyetheretherketone, silicone, or the like or combinations thereof). In at least some embodiments, the helical member 556 is formed from a polymer monofilament.

The helical member 556 can be attached to the outer surface 408 of the lead 303 using any suitable technique including, for example, reflowing, chemical bonding, using one or more adhesives, or the like or combinations. Reflowing involves heating material (using material from the body of the lead, the helical member, another electrically nonconductive material (e.g., one or more polymers), or some combination thereof) enough to enable the materials to meld together and allowing the melded materials to cool enough to set.

In at least some embodiments, the outer surface 408 of the lead 303, or the one or more features of the anchoring arrangement, or both, is roughened to increase the coefficient of friction. The lead, feature(s), or both, may be roughened using any suitable technique including, for example, chemical etching, laser etching, mechanical abrading, or the like.

It will be understood that, unless otherwise indicated, any of the disclosed features of the anchoring arrangement (see e.g., FIGS. 5A-10), including the helical members (see e.g., FIGS. 5A-6D and FIGS. 8-10), may be formed from any of the above-mentioned materials and may be attached to their respective leads using any of the above-mentioned techniques.

Turning to FIG. 5B, in at least some embodiments the helical member includes one or more anchor conductors. The one or more anchor conductors may be embedded in one or more layers of electrically nonconductive material. In at least some embodiments, the anchor conductors extend along a length of the helical members and electrically couple electrodes to terminals (e.g., via welding, crimping, or the like). As shown in FIG. 5B, in some embodiments the anchor conductor is used to replace one or more lead conductors 454. As shown in FIG. 5C, in some embodiments the anchor conductors are used to electrically couple additional electrodes to additional terminals disposed along the lead.

FIG. 5B illustrates, in schematic side view, another embodiment of the anchoring arrangement 502 disposed along the lead 303. The lead 303 includes the electrode 334 and the terminal 344. In FIG. 5B, the helical member 556 is shown including an anchor conductor 566 coupling the electrode 334 to the terminal 344. In at least some embodiments, the helical member 556 and corresponding anchor conductor 566 extend between the electrode 334 and the terminal 344 entirely external to the outer surface 408 of the lead 303. In at least some embodiment, the electrode 334 is electrically coupled to the terminal 344 solely via the anchor conductor 566. In at least some embodiments there are no lead conductors disposed beneath the outer surface 408 of the lead 303.

It will be understood that, in the case of leads with multiple electrodes coupled to multiple terminals, in at least some embodiment each of the electrodes is electrically coupled to each of the terminals solely via one or more anchor conductors. In at least some embodiments, the helical member(s) and corresponding anchor conductor(s) extending between the electrodes and terminals of the leads with multiple electrodes/terminals extend entirely external to the outer surface of the lead. In at least some embodiments, the leads with multiple electrodes/terminals have no lead conductors disposed beneath outer surfaces of those leads.

FIG. 5C illustrates, in schematic side view, another embodiment of the anchoring arrangement 502 disposed along the lead 303. The lead 303 includes first and second electrodes 334a and 334b, respectively, and first and second terminals 344a and 344b, respectively. In FIG. 5C, the lead conductor 454 is shown electrically coupling the first electrode 334a to the first terminal 344a, and the anchor conductor 566 of the helical member 506 is shown electrically coupling the second electrode 334b to the second terminal 344b. In at least some embodiments, the helical member 556 (and the corresponding anchor conductor 566 of the helical member 556) extends between the second electrode 334b and the second terminal 344b entirely external to the outer surface 408 of the lead 303.

Turning to FIG. 6A, in at least some embodiments the anchoring arrangement is disposed along a lead configured for long-term (e.g., more than six months) implantation, such as a percutaneous spinal cord stimulation lead. The lead can include any suitable number of electrodes including, for example, one, two, three, four, five, six, seven, eight, twelve, sixteen, twenty, twenty-four, thirty-two, sixty-four, or more electrodes.

FIG. 6A illustrates, in schematic side view, one embodiment of the anchoring arrangement 502 disposed along a lead 603 having a body 606 with an outer surface 608, a distal portion 610, and an opposing proximal portion 612. The anchoring arrangement 502 includes a helical member 656 extending along the outer surface 608 of the lead 603.

The lead 603 includes three electrodes 634a, 634b, and 634c disposed along the distal portion 610 of the lead 603 and coupled to three terminals 644a, 644b, and 634c, respectively, disposed along the proximal portion 612 of the lead 603 via three lead conductors 654a, 654b, and 634c, respectively. In at least some embodiments, the lead conductors 654a, 65b, and 634c each extend between the electrodes 634a, 634b. and 634c and the terminals 644a, 644b, and 634c entirely beneath the outer surface 608 of the lead 603.

Turning to FIG. 6B, in at least some embodiments the helical member includes one or more anchor conductors. The one or more anchor conductors may be encased in one or more layers of electrically nonconductive material. In at least some embodiments, the one or more anchor conductors electrically couple electrodes to terminals. As shown in FIG. 6B, in some embodiments one of the lead conductors 654a, 654b, or 634c is replaced by the anchor conductor. As shown in FIG. 6C, in some embodiments two or more of the lead conductors 654a, 654b, or 634c are replaced by one or more anchor conductors disposed along a single helical member. As shown in FIG. 6D, in some embodiments two or more of the lead conductors 654a, 654b, or 634c are replaced by two or more anchor conductors disposed along two or more helical members.

FIG. 6B illustrates, in schematic side view, another embodiment of the anchoring arrangement 502 disposed along the lead 603. The anchoring arrangement 502 includes an anchor conductor 666 disposed along the helical member 656. The anchor conductor electrically couples the electrode 634c to the terminal 644c. The remaining electrodes 634a and 634b are coupled to the remaining terminals 644a and 644b, respectively, via the lead conductors 654a and 654b, respectively.

FIG. 6C illustrates, in schematic side view, yet another embodiment of the anchoring arrangement 502 disposed along the lead 603. The anchoring arrangement 502 includes two anchor conductors 666a and 666b disposed along the helical member 656. The anchor conductor 666a electrically couples the electrode 634a to the terminal 644a and the anchor conductor 666b electrically couples the electrode 634c to the terminal 644c. The remaining electrode 634b is coupled to the remaining terminal 644b via the lead conductor 654a.

It will be understood that the helical member 656 may include any suitable number of anchor conductors. In at least some embodiments, a single anchor conductor electrically couples multiple electrodes to one or more of the terminals. In at least some embodiments, each of the multiple electrodes of the lead is electrically coupled to each of the terminals of the lead via anchor conductors extending along a single helical member disposed over the outer surface 608 of the lead 603.

FIG. 6D illustrates, in schematic side view, still yet another embodiment of an anchoring arrangement 502 disposed along the lead 603. The anchoring arrangement 502 includes two helical members 656a and 656b. Anchor conductor 666a is disposed along the helical member 656a, and anchor conductor 666b is disposed along the helical member 656b. The anchor conductor 666a electrically couples the electrode 634c to the terminal 644c, and the anchor conductor 666b electrically couples the electrode 634b to the terminal 644b. The remaining electrode 634a is coupled to the remaining terminal 644a via the lead conductor 654a.

In FIG. 6B, two anchor conductors are disposed along the helical member 656. It will be understood that any suitable number of anchor conductors may be disposed along any particular helical member of the anchoring arrangement including, for example, one, two, three, four, five, six, seven, eight, nine, ten, or more anchor conductors. The number of anchor conductors disposed along any particular helical member can be equal to, greater than, or fewer than the number of electrodes disposed along the lead.

The anchoring arrangement can include any suitable number of helical members including, for example, one, two, three, four, five, six, seven, eight, twelve, sixteen, twenty, twenty-four, thirty-two, sixty-four, or more. In some embodiments, the number of helical members is equal to the number of electrodes disposed along the lead. In other embodiments, then number of helical members is greater than or fewer than the number of electrodes disposed along the lead. In at least some embodiments, when the anchoring arrangement includes multiple helical members, at least one of the helical members includes a different number of anchor conductors than at least one other of the helical members.

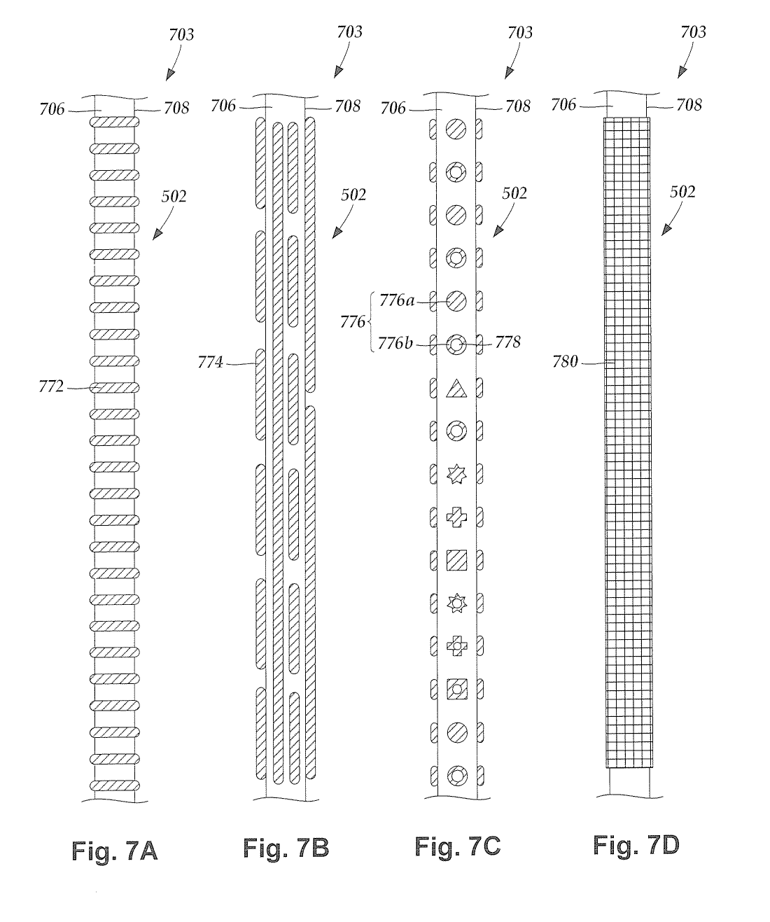

Turning to FIG. 7A, in at least some embodiments the anchoring arrangement includes one or more different anchoring elements disposed over the outer surface of the lead in addition to, or in lieu of, one or more helical members. The anchoring elements shown in FIGS. 7A-7D are shown extending along an intermediate portion of the lead. It will be understood that, as with the helical members, and as discussed below with reference to FIG. 9, the anchoring elements of the anchoring arrangement can be disposed along any portion(s) of the lead.

In at least some embodiments, the anchoring arrangement includes one or more rings extending around a circumference of the lead. FIG. 7A illustrates, in schematic side view, another embodiment of the anchoring arrangement 502 disposed along an intermediate portion of a lead 703 having a body 706 with an outer surface 708. The anchoring arrangement 502 includes multiple longitudinally-spaced-apart rings, such as ring 772, disposed along the outer surface 708 of the lead 703, and projecting outwardly therefrom. In some embodiments, the rings 772 form continuous loops of material extending completely around a circumference of the lead 703. In other embodiments, the rings 772 are open-looped, or C-shaped, and extend around at least 80%, 90%, or 95% or more of the circumference (or a perimeter) of the lead 703. The anchoring arrangement 502 can include any suitable number of rings 772 including, for example, one, two, three, four, five, six, seven, eight, nine, ten, eleven, twelve, fourteen, sixteen, twenty, twenty-five, thirty, forty, fifty, or more rings 772.

FIG. 7B illustrates, in schematic side view, another embodiment of the anchoring arrangement 502 disposed along the intermediate portion of the lead 703. The anchoring arrangement 502 includes one or more strips, such as strip 774, which extend along the outer surface 708 of the lead 703 and project outwardly therefrom. The strips 774 can extend along any suitable portion of the lead 703 and be any suitable length. In at least some embodiments, the strips 774 make less than one revolution about the lead 703. In at least some embodiments, the strips 774 are circumferentially-spaced-apart strips from one another along the lead 703. In at least some embodiments, the strips 774 extend longitudinally along the outer surface 708 of the lead 703.

Any suitable number of strips 774 may be disposed around a circumference of the lead 703 including, for example, one, two, three, four, five, six, seven, eight, or more strips 774. Any suitable number of strips 774 may be disposed along the longitudinal length of the lead 703 at any particular circumferential position of the lead including, for example, one, two, three, four, five, six, seven, eight, or more strips 774. In at least some embodiments, the anchoring arrangement includes at least one strip 774 that extends along the entire longitudinal length of the lead between the proximal-most electrode and the distal-most terminal.

FIG. 7C illustrates, in schematic side view, another embodiment of the anchoring arrangement 502 disposed along the intermediate portion of the lead 703. The anchoring arrangement 502 includes one or more knobs 776 disposed along an outer surface 708 of the lead 703 and projecting outwardly therefrom. The knobs 776 can be any suitable shape including, for example, round, oval, triangular, rectangular, cruciform, star-shaped, or the like. In at least some embodiments, at least one of the knobs 776 defines at least one aperture 778. In at least some embodiments, the anchoring arrangement 502 includes at least one knob 776 that has a shape that is different from at least one other knob 776.

Any suitable number of knobs 776 may be disposed around a circumference of the lead 703 including, for example, one, two, three, four, five, six, seven, eight, or more knobs 776. Any suitable number of knobs 776 may be disposed along any the longitudinal length of the lead 703 at any particular circumferential position of the lead including, for example, one, two, three, four, five, six, seven, eight, ten, fifteen, twenty, or more knobs 776.

FIG. 7D illustrates, in schematic side view, another embodiment of the anchoring arrangement 502 disposed along the intermediate portion of the lead 703. The anchoring arrangement 502 includes a meshed material 780 disposed over a portion of an outer surface 708 of the lead 703. The anchoring arrangement 502 may include either a single piece of meshed material 780, or multiple pieces of meshed material 780. In at least some embodiments, the meshed material 780 is tubular.

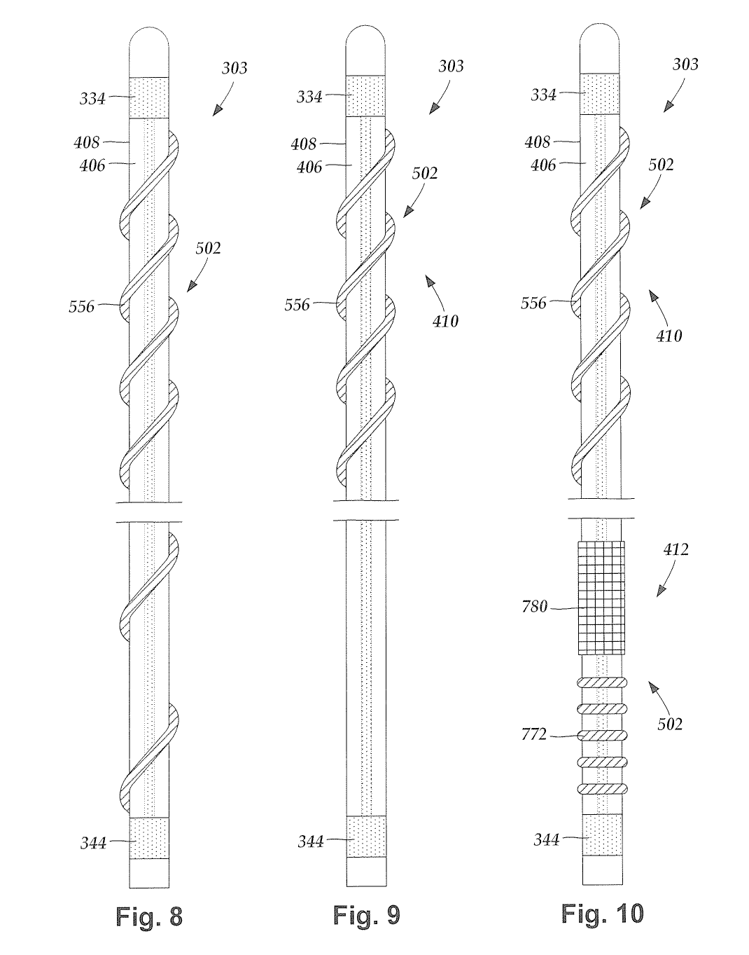

Turning to FIG. 8, the anchoring arrangements described herein, and shown in FIGS. 5A-7D, may extend along their respective leads in either a constant pitch or a variable pitch. FIG. 8 illustrates, in schematic side view, one embodiment of the anchoring arrangement 502 disposed along the lead 303. The anchoring arrangement 502 includes the helical member 556 extending along the outer surface 408 of the lead 303. In FIG. 8, the helical member 556 is shown having a variable pitch such that the distal portion of the helical member 556 has a tighter pitch than the proximal portion of the helical member 556.

In alternate variably-pitched embodiments, the relatively more tightly-pitched region of the one or more anchoring arrangements is disposed along an intermediate portion, or the proximal portion of the lead. In at least some embodiments, the one or more anchoring arrangements include multiple regions that are more tighter-pitched than other regions of the one or more anchoring arrangements.

Turning to FIG. 9, the anchoring arrangements described herein, and shown in FIGS. 5A-7D, may extend exclusively along one or more particular portions of the lead. FIG. 9 illustrates, in schematic side view, one embodiment of the anchoring arrangement 502 disposed along the lead 303. The anchoring arrangement 502 includes the helical member 556 extending along the outer surface 408 of the lead 303. In FIG. 9, the helical member 556 is shown extending exclusively along the distal portion 410 of the lead 403. In alternate embodiments, the one or more anchoring arrangements extend exclusively along the proximal portion 412 or an intermediate portion of the lead 303.

In at least some embodiments, the one or more anchoring arrangements extend discontinuously along at least one of the proximal portion 412 or an intermediate portion of the lead 303 in addition to, or in lieu of, extending along the distal portion of the lead. In some embodiments, at least one of the anchoring arrangements extends along at least 30%, 40%, 50%, 60%, 70%, 80%, 90%, or 100% of the longitudinal length of the lead. In some embodiments, at least one of the anchoring arrangements extends along no more than 80%, 70%, 60%, 50%, 40%, 30%, 70%, or 10% of the longitudinal length of the lead.

In some embodiments, at least one of the anchoring arrangements extends exclusively between the proximal-most electrode and the distal-most terminal. In some embodiments, at least one of the anchoring arrangements extends along at least 50%, 60%, 70%, 80%, 90%, 100% of the longitudinal length of the lead between the proximal-most electrode and the distal-most terminal.

In some embodiments, the one or more anchoring arrangements extend exclusively distal to the distal-most electrode, exclusively distal to the proximal-most electrode, exclusively proximal to the proximal-most terminal, exclusively proximal to the distal-most terminal, or some combination thereof. In some embodiments, the one or more anchoring arrangements extend exclusively along the electrodes and any portion(s) of the lead disposed between adjacent electrodes. In some embodiments, the one or more anchoring arrangements extend exclusively along the terminals and any portion(s) of the lead between adjacent terminals. In some embodiments, the one or more anchoring arrangements extend along the distal portion of the lead exclusively proximal to the proximal-most electrode. In some embodiments, the one or more anchoring arrangements extend along the proximal portion of the lead exclusively distal to the distal-most electrode.

Turning to FIG. 10, in at least some embodiments the anchoring arrangement includes multiple different types of anchoring elements disposed along the lead. In at least some embodiments, the anchoring arrangement includes multiple different types of anchoring elements in combination with one or more helical members. FIG. 10 illustrates, in schematic side view, one embodiment of the anchoring arrangement 502 extending along the outer surface 408 of the lead 303. The anchoring arrangement 502 includes each of the helical member 556, longitudinally-spaced-apart rings 772, and the meshed material 780. In FIG. 9, the helical member 556 is shown extending exclusively along the distal portion 410 of the lead 403, while the longitudinally-spaced-apart rings 772 and the meshed material 780 are shown extending exclusively along the proximal portion 412 of the lead 403.

It will be understood that the anchoring arrangement may include any combination of the disclosed anchoring elements/helical members. It will additionally be understood that, when the anchoring arrangements includes multiple different types of anchoring elements, helical members, or combinations thereof, these features may be arranged in any desired order along the longitudinal length of the lead in any suitable pitch, or combination of pitches.

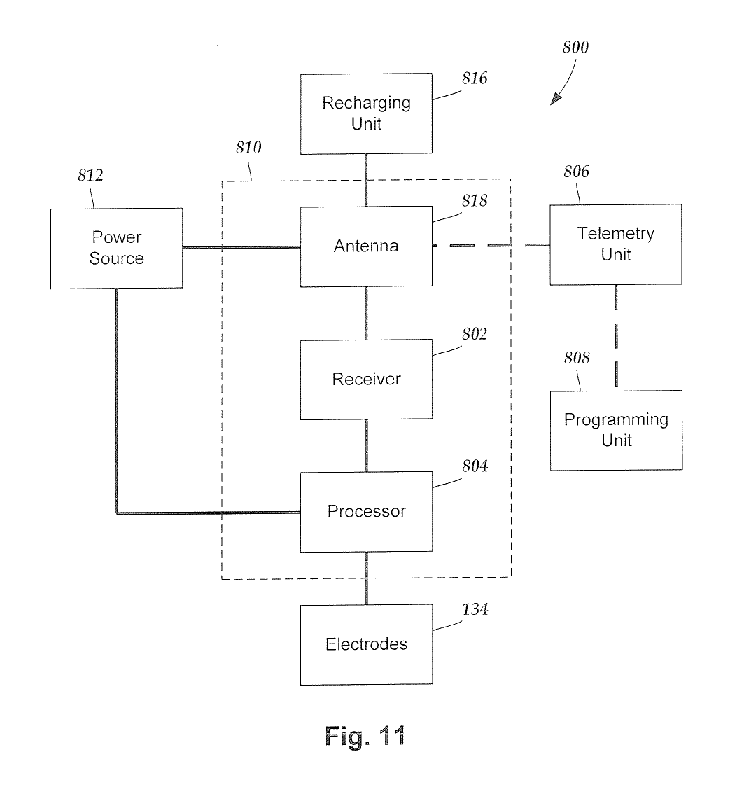

FIG. 11 is a schematic overview of one embodiment of components of an electrical stimulation system 1100 including an electronic subassembly 1110 disposed within a control module. It will be understood that the electrical stimulation system can include more, fewer, or different components and can have a variety of different configurations including those configurations disclosed in the stimulator references cited herein.

Some of the components (for example, a power source 1112, an antenna 1118, a receiver 1102, and a processor 1104) of the electrical stimulation system can be positioned on one or more circuit boards or similar carriers within a sealed housing of an implantable pulse generator, if desired. Any power source 1112 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193 incorporated herein by reference.

As another alternative, power can be supplied by an external power source through inductive coupling via the optional antenna 1118 or a secondary antenna. The external power source can be in a device that is mounted on the skin of the user or in a unit that is provided near the user on a permanent or periodic basis.

If the power source 1112 is a rechargeable battery, the battery may be recharged using the optional antenna 1118, if desired. Power can be provided to the battery for recharging by inductively coupling the battery through the antenna to a recharging unit 1116 external to the user. Examples of such arrangements can be found in the references identified above.

In one embodiment, electrical current is emitted by the electrodes 134 on the paddle or lead body to stimulate nerve fibers, muscle fibers, or other body tissues near the electrical stimulation system. The processor 1104 is generally included to control the timing and electrical characteristics of the electrical stimulation system. For example, the processor 1104 can, if desired, control one or more of the timing, frequency, strength, duration, and waveform of the pulses. In addition, the processor 1104 can select which electrodes can be used to provide stimulation, if desired. In some embodiments, the processor 1104 selects which electrode(s) are cathodes and which electrode(s) are anodes. In some embodiments, the processor 1104 is used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 1108 that, for example, allows modification of pulse characteristics. In the illustrated embodiment, the processor 1104 is coupled to a receiver 1102 which, in turn, is coupled to the optional antenna 1118. This allows the processor 1104 to receive instructions from an external source to, for example, direct the pulse characteristics and the selection of electrodes, if desired.

In one embodiment, the antenna 1118 is capable of receiving signals (e.g., RF signals) from an external telemetry unit 1106 which is programmed by the programming unit 1108. The programming unit 1108 can be external to, or part of, the telemetry unit 1106. The telemetry unit 1106 can be a device that is worn on the skin of the user or can be carried by the user and can have a form similar to a pager, cellular phone, or remote control, if desired. As another alternative, the telemetry unit 1106 may not be worn or carried by the user but may only be available at a home station or at a clinician's office. The programming unit 1108 can be any unit that can provide information to the telemetry unit 1106 for transmission to the electrical stimulation system 1100. The programming unit 1108 can be part of the telemetry unit 1106 or can provide signals or information to the telemetry unit 1106 via a wireless or wired connection. One example of a suitable programming unit is a computer operated by the user or clinician to send signals to the telemetry unit 1106.

The signals sent to the processor 1104 via the antenna 1118 and the receiver 1102 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 1100 to cease operation, to start operation, to start charging the battery, or to stop charging the battery. In other embodiments, the stimulation system does not include the antenna 1118 or receiver 1102 and the processor 1104 operates as programmed.

Optionally, the electrical stimulation system 1100 may include a transmitter (not shown) coupled to the processor 1104 and the antenna 1118 for transmitting signals back to the telemetry unit 1106 or another unit capable of receiving the signals. For example, the electrical stimulation system 1100 may transmit signals indicating whether the electrical stimulation system 1100 is operating properly or not or indicating when the battery needs to be charged or the level of charge remaining in the battery. The processor 1104 may also be capable of transmitting information about the pulse characteristics so that a user or clinician can determine or verify the characteristics.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. A lead assembly comprising:
an electrical stimulation lead comprising
lead body having a distal portion, a proximal portion, a longitudinal length, and an outer surface,
at least one electrode disposed along the distal portion of the lead body,
at least one terminal disposed along the proximal portion of the lead body, and
at least one lead conductor electrically coupling the at least one electrode to the at least one terminal; and
an anchoring arrangement configured and arranged to reduce undesired movement of the lead relative to a patient when the lead is inserted into the patient, the anchoring arrangement comprising at least one helical member attached to, and projecting outwardly from, the outer surface of the lead, the at least one helical member extending along at least 30% of the longitudinal length of the lead and making at least one full coil around the lead.

2. The lead assembly of claim 1, wherein the at least one helical member extends along the entire longitudinal length of the lead between the at least one electrode and the at least one terminal.

3. The lead assembly of claim 1 wherein the at least one helical member extends exclusively along the distal portion of the lead.

4. The lead assembly of claim 1, wherein the at least one helical member is formed entirely from electrically nonconductive polymer.

5. The lead assembly of claim 1, wherein the at least one helical member comprises a first anchor conductor encased in electrically nonconductive material.

6. The lead assembly of claim 5, wherein the at least one electrode comprises a first electrode and a second electrode, wherein the at least one terminal comprises a first terminal and a second terminal, wherein the first electrode is coupled to the first terminal via the at least one lead conductor, and wherein the second electrode is coupled to the second terminal via the first anchor conductor.

7. The lead assembly of claim 5, wherein the at least one helical member further comprises a second anchor conductor encased in the electrically nonconductive material.

8. The lead assembly of claim 7, wherein the at least one helical member comprises a single helical member, and wherein the first anchor conductor and the second anchor conductor are both disposed along the single helical member.

9. The lead assembly of claim 7, wherein the at least one helical member comprises a first helical member and a second helical member, and wherein the first anchor conductor is disposed along the first helical member and the second anchor conductor is disposed along the second helical member.

10. The lead assembly of claim 7, wherein the at least one electrode further comprises a third electrode and the at least one terminal further comprises a third terminal, and wherein the second anchor conductor couples the third electrode to the third terminal.

11. The lead assembly of claim 1, wherein the at least one helical member has a constant pitch.

12. The lead assembly of claim 1, wherein the at least one helical member has a variable pitch.

13. The lead assembly of claim 1, wherein the anchoring arrangement further comprises a meshed material disposed along a portion of the longitudinal length of the lead body.

14. The lead assembly of claim 1, wherein the anchoring arrangement further comprises at least one of an outwardly-projecting knob or a longitudinal strip attached to, and extending along, the outer surface of the lead.

15. An electrical stimulating system comprising:
the lead assembly of claim 1;
a control module coupleable to the electrical stimulation lead of the lead assembly, the control module comprising
a housing, and
an electronic subassembly disposed in the housing; and
a connector for receiving the electrical stimulation lead, the connector comprising
a connector housing defining a port configured and arranged for receiving the proximal portion of the lead body of the electrical stimulation lead, and
at least one connector contact disposed in the connector housing, the at least one connector contact configured and arranged to couple to the at least one terminal of the electrical stimulation lead when the proximal portion of the electrical stimulation lead is received by the port.

16. A method of forming the electrical stimulation lead of claim 1 the method comprising:
disposing a plurality of electrodes along a distal portion of a lead body, the plurality of electrodes comprising a first electrode;
disposing a plurality of terminals along a proximal portion of the lead body, the plurality of terminals comprising a first terminal;
electrically coupling the first electrode to the first terminal using a lead conductor extending along a longitudinal length of the lead within an outer surface of the lead; and
attaching a helical member to the outer surface of the lead, the helical member extending along at least 30% of the longitudinal length of the lead and making at least one full coil around the lead.

17. The method of claim 16, wherein attaching helical member to the outer surface of the lead comprises at least one of reflowing or chemically bonding the helical member to the lead.

18. The method of claim 16, further comprising electrically coupling a second electrode of the plurality of electrodes to a second terminal of the plurality of terminals using an anchor conductor disposed in the helical member.

19. A lead assembly comprising:
an electrical stimulation lead comprising
a lead body having a distal portion, a proximal portion, a longitudinal length, and an outer surface,
at least one electrode disposed along the distal portion of the lead,
at least one terminal disposed along the proximal portion of the lead, and
at least one lead conductor electrically coupling the at least one electrode to the at least one terminal; and
an anchoring arrangement configured and arranged to reduce undesired movement of the lead relative to a patient when the lead is inserted into the patient, the anchoring arrangement comprising a plurality of anchoring elements attached to, and projecting outwardly from, the outer surface of the lead, the plurality of anchoring elements comprising
a proximal-most element,
a distal-most element, and
a plurality of intermediate elements disposed between the proximal-most element and the distal-most element,
wherein at least 30% of the longitudinal length of the lead body is disposed between the proximal-most element and the distal-most element,
wherein the plurality of anchoring elements include at least one of a ring, a strip, or a knob.

20. A lead assembly comprising:
an electrical stimulation lead comprising
a lead body having a distal portion, a proximal portion, a longitudinal length, and an outer surface,
at least one electrode disposed along the distal portion of the lead,
at least one terminal disposed along the proximal portion of the lead, and
at least one lead conductor electrically coupling the at least one electrode to the at least one terminal; and
an anchoring arrangement configured and arranged to reduce undesired movement of the lead relative to a patient when the lead is inserted into the patient, the anchoring arrangement comprising a meshed material disposed over, and attached to, the outer surface of the lead, the meshed material extending along at least 30% of the longitudinal length of the lead.
